(19) United States
(12) Patent Application Publication
(10) Pub. No.: US 2026/0325482 A1
(43) Pub. Date: Oct. 22, 2026

(54) TASIMELTEON FOR USE IN THE TREATMENT OF DELAYED SLEEP-WAKE PHASE DISORDER

(71) Applicant: VANDA PHARMACEUTICALS INC., Washington, DC (US)

(72) Inventors: Sandra P. SMIESZEK, Washington, DC (US); Mihael H. POLYMEROPOULOS, Washington, DC (US)

(21) Appl. No.: 19/491,466

(22) PCT Filed: Jun. 10, 2024
(86) PCT No.: PCT/US2024/033256

§ 371 Date: Dec. 9, 2025

Related U.S. Application Data
(60) Provisional application No. 63/507,727, filed on Jun. 12, 2023.

(51) Int. Cl. A61K 31/343

(52) U.S. Cl. CPC A61K 31/343

(57) ABSTRACT

The invention relates generally to circadian rhythm disorders and sleep disorders and, more particularly, to the treatment of such disorders based on a patient's OPN4 genotype.

### CROSS-REFERENCE TO RELATED APPLICATIONS

This application claims the benefit of co-pending U.S. Provisional Patent Application Ser. No. 63/507,727, filed 12 Jun. 2023, which is hereby incorporated herein as though fully set forth.

### BACKGROUND

### Melanopsin

Melanopsin (OPN4) is a blue light-sensitive opsin-type G-protein coupled receptor. It is highly expressed in photosensitive retinal ganglion cells (RGCs), which mediate responses to light, including the regulation of sleep, circadian photoentrainment, and pupillary light response.

The OPN4 gene is approximately 11.8 kb, including nine introns and ten exons, and is mapped to the long arm of chromosome 10. When activated by light, an action potential is triggered in photosensitive RGCs that travels to targets in the brain, such as the olivary pretectal nucleus (OPN). Melanopsin stimulation ultimately mediates physiological responses to light, including pupil constriction and the inhibition of melatonin release from the pineal gland.

The suprachiasmatic nucleus (SCN) of the hypothalamus maintains circadian rhythm and receives input from the eye's rods, cones, and photosensitive RGCs. In mammals, melanopsin interacts with the SCN through the retinohypothalamic tract (RHT). Studies have shown that melanopsin knockout mice have reduced and delayed photoentrainment, whereas knockout mice that also lacked rods and cones lost all photoentrainment. This suggests that all three photopigments—melanopsin, rhodopsin (produced in rods), and photopsin (produced in cones)—are involved in photoentrainment of circadian rhythms.

In humans, mutations in the OPN4 gene have been associated with clinical disorders, including seasonal affective disorder.

### Tasimelteon

As a circadian regulator, tasimelteon is a dual melatonin receptor agonist approved in the United States under the trade name HETLIOZ® for the treatment of Non-24-Hour Sleep-Wake Disorder (Non-24) and for the treatment of Nighttime Sleep Disturbances in Smith-Magenis Syndrome (SMS). Tasimelteon was shown to be safe and well-tolerated in previous clinical studies in healthy volunteers, in participants with primary insomnia, in participants with Non-24, and in pediatric participants with SMS.

Tasimelteon has a greater affinity for the MT2 receptor than the MT1 receptor. For doses ranging from 3 mg to 300 mg, the pharmacokinetics of tasimelteon are linear. Tasimelteon has an absolute bioavailability of 38% and its peak plasma concentration (tₘₐₓ) occurs approximately 30 minutes to three hours after fasted oral administration.

Liquid formulations of tasimelteon and methods for their administration are described in International Patent Application Publication No. WO/2021/119456, which is hereby incorporated herein as though fully set forth.

### SUMMARY

In one embodiment, the invention provides, in a method of treating a patient for a circadian rhythm disorder or a sleep disorder, an improvement comprising: selecting as said patient an individual whose genotype includes a loss-of-function variant of the OPN4 gene.

In another embodiment, the invention provides a method of treating a patient having a circadian rhythm disorder or a sleep disorder, the method comprising: administering to said patient an amount of tasimelteon effective to treat said circadian rhythm disorder or sleep disorder, wherein the patient has been determined to have a loss-of-function variant of the OPN4 gene.

### DETAILED DESCRIPTION

A carrier of the rare (gnomAD MAF 0.0002) rs143641898 variant (OPN4:NM_033282:exon4:c.C502T:p. R168C) is identified among patients exhibiting symptoms of delayed sleep-wake phase disorder (DSWPD). The rs143641898 variant is a highly conserved missense variant predicted to be damaging. It is part of the E/DRY motif found in nearly all G protein-coupled receptors.

Sleep onset in the patient is consistent and significantly delayed in comparison to a healthy control population (n=300). Salivary melatonin assay confirms dim light melatonin on set (DLMO) at 23:20. The patient has no other known loss-of-function mutations in the core clock genes.

The rs143641898 variant is tested using an in vitro expression system as part of a study to determine the functional phenotypes of missense OPN4 variants. Sixteen potentially deleterious variants are selected for functional characterization using calcium imaging of melanopsin-driven light responses in HEK293T cells.

The rs143641898 variant is shown to be incapable of binding retinal chromophore, suggesting that it renders the OPN4 protein non-functional. The introduction of the rs143641898 variant protein (R168C) abolishes responses to light.

This discovery is useful in both the identification of individuals with disrupted melanopsin-based light perception and the treatment or prevention of attendant disease, such as DSWPD or insomnia, in such individuals. Treatment or prevention of such disease may include, for example, the administration of tasimelteon to a patient. Tasimelteon may be administered at a dose from 3 mg/d to 300 mg/d, e.g., 10 mg/d to 100 mg/d, 20 mg/d to 50 mg/d, etc. A daily dose of 20 mg administered once, before bedtime, has been shown to be effective in treating Non-24. A daily dose of 0.7 mg/kg administered once daily has been shown to be effective in treating SMS in patients having a body mass of 28 kg or less.

The timing and manner of administration of tasimelteon can be important in the efficacy of treating circadian rhythm disorders or sleep disorders. High-calorie/high-fat meals have been found to significantly lower tasimelteon's Cₘₐₓ and delay its Tₘₐₓ, although the extent of absorption is not significantly affected. As such, it is recommended that tasimelteon be taken without food (e.g., administered without food and with no food for at least 30 minutes prior to administration, preferably with no food for at least one hour prior to administration, more preferably with no food for at least one-and-one-half hours prior to administration, still more preferably with no food for at least two hours prior to administration, even more preferably with no food for at least two-and-one-half hours prior to administration, most preferably with no food for at least three hours prior to administration).

Similarly, tasimelteon is recommended to be administered once daily, at least 30 minutes before a target bedtime, e.g., from about 30 minutes to about 90 minutes before the target bedtime, or about one hour before the target bedtime.

As will be appreciated by one skilled in the art, the particular dose, timing, and other conditions of the administration of tasimelteon may vary based on a patient's age, body mass, the condition to be treated and its severity, co-morbidities, co-administered drugs, etc.

Overall treatment efficacy may be improved, for example, by selecting for treatment an individual whose genotype includes a loss-of-function variant of the OPN4 gene, such as the rs143641898 variant. As will be recognized by one skilled in the art, however, it is possible that other variants of the OPN4 gene could result in such loss-of-function, including other variants resulting in the R168C variant protein.

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. In a method of treating a patient for a circadian rhythm disorder or a sleep disorder, the improvement comprising:
selecting as said patient an individual whose genotype includes a loss-of-function variant of the OPN4 gene.

2. The improvement of claim 1, wherein the loss of function variant results in a substitution of cysteine for arginine at position 168 of the OPN 4 protein.

3. The improvement of claim 2, wherein the loss-of-function variant is rs143641898.

4. The improvement of claim 1, wherein the circadian rhythm disorder is delayed sleep-wake phase disorder.

5. The improvement of claim 1, wherein the sleep disorder is insomnia.

6. The improvement of claim 1, wherein treating the patient includes administering tasimelteon to the patient.

7. The improvement of claim 6, wherein treating the patient includes administering 20 mg of tasimelteon to the patient once daily before bedtime.

8. The improvement of claim 7, wherein the tasimelteon is administered at least about 30 minutes before bedtime.

9. The improvement of claim 8, wherein the tasimelteon is administered without food and with no food for at least about 30 minutes prior to administration.

10. The improvement of claim 1, further comprising:
determining that the individual's genotype includes the loss-of-function variant.

11. A method of treating a patient having a circadian rhythm disorder or a sleep disorder, the method comprising:
administering to said patient an amount of tasimelteon effective to treat said circadian rhythm disorder or sleep disorder,
wherein the patient has been determined to have a loss-of-function variant of the OPN4 gene.

12. The method of claim 11, wherein the loss-of-function variant results in a substitution of cysteine for arginine at position 168 of the OPN4 protein.

13. The method of claim 12, wherein the loss-of-function variant is rs143641898.

14. The method of claim 11, wherein the circadian rhythm disorder is delayed sleep-wake phase disorder.

15. The method of claim 11, wherein the sleep disorder is insomnia.

16. The method of claim 11, wherein the amount of tasimelteon effective to treat the circadian rhythm disorder or sleep disorder is 20 mg/day.

17. The method of claim 16, wherein the tasimelteon is administered once daily at least about 30 minutes before bedtime.

18. The method of claim 17, wherein the tasimelteon is administered without food and with no food for at least about 30 minutes prior to administration.

19. The method of claim 17, further comprising:
determining that the patient's genotype includes the loss-of-function variant of the OPN4 gene.
